(19) United States
(12) Patent Application Publication
(10) Pub. No.: US 2023/0258906 A1
(43) Pub. Date: Aug. 17, 2023

(54) AUTOMATIC AND MANUAL ZOOM MECHANISM FOR SLIT LAMP-BASED DRY EYE INSPECTION INSTRUMENT

(71) Applicant: CHONGQING KANGHUA RUIMING SCIENCE TECHNOLOGY CO., LTD, Chongqing (CN)

(72) Inventor: Yi Wang, Chongqing (CN)

(21) Appl. No.: 18/004,052

(22) PCT Filed: Dec. 29, 2021
(86) PCT No.: PCT/CN2021/142600

§ 371 Date: Dec. 31, 2022

(30) Foreign Application Priority Data
Nov. 3, 2020 (CN) 202011209100.7

(51) Int. Cl. G02B 7/10; A61B 3/135; A61B 3/10

(52) U.S. Cl. CPC G02B 7/10; A61B 3/101; A61B 3/135

(57) ABSTRACT

An automatic and manual zooming mechanism based on ophthalmic slit lamp with dry eye inspection, characterized in that it comprises an optical body zoom drum seat, an optical body zoom drum, a zoom handwheel and an electric drive mechanism; The doubling drum is installed in the optical body zooming drum seat, and the two ends of the optical body zooming drum are respectively connected with zooming handwheels through connecting shafts; on the optical body zooming drum seat An electric drive mechanism is provided, and the electric drive mechanism is used to drive the optical body variable magnification drum to rotate. First, the structure is simple, and it is convenient to realize the automatic zoom function of the slit lamp of the Galileo optical system and the slit lamp type dry eye inspection instrument; second, the automatic zoom function of the slit lamp of the Galileo optical system is realized, while the manual zoom function is also retained. Third, it is convenient to realize the intelligent operation and remote operation of the entire slit lamp and the slit lamp type dry eye inspection instrument.

### TECHNICAL FIELD

The invention relates to the field of medical equipment, in particular to an automatic and manual zoom mechanism based on a slit lamp dry eye inspection instrument.

### BACKGROUND TECHNIQUE

The eyes are the most important organ of the human senses, and about 80% of the knowledge in the brain is acquired through the eyes. Reading and recognizing characters, seeing pictures and pictures, seeing people, and enjoying beautiful scenery all use the eyes.

Dry eye is the most common and high incidence ocular surface disease. Its incidence rate in China is 20 \~36%. In recent years, its development trend has shown an obvious growth trend due to the increase of eye use frequency and the increase of eye time. Research on this disease is of practical significance to human health.

Various examinations of dry eye, including: tear river height measurement, tear film break-up time measurement, meibomian gland analysis, lipid layer analysis, eye margin analysis, eye redness analysis, etc.

There are three types of instruments that can complete the above dry eye examination items: the first type is the instrument developed on the basis of the original corneal topograph; the second type is the instrument developed on the basis of the original slit lamp, with additional corneal projection for dry eye examination. Device; the third type of instrument for dry eye inspection, it can only inspect dry eye items and has no other functions.

This patent is an improvement on the dry eye examination instrument developed on the basis of the second type, the slit lamp.

Slit lamp microscope (slit lamp for short) is an indispensable and important instrument for ophthalmic examination, and it is also the most widely used basic instrument for ophthalmic routine examination..

The structure of the slit lamp is mainly composed of four parts: the slit illumination mechanism, the microscope optical body, the moving platform mechanism, and the chin rest mechanism. The optical system of the microscope optical body adopts the ordinary crossed binocular optical system and the Galileo optical system. Slit lamp type dry eye examination instrument using Galileo optics.

The Galileo optical system is a parallel optical system. Its optical path is based on the principle of parallelism. The use of two parallel beams of light with no difference in the optical path can greatly improve the visibility of the image.

The Galileo optical system used in the slit lamp body (FIG. 7 ) is composed of a large objective lens assembly, a variable magnification objective lens assembly, and an eyepiece assembly. Different magnification ratios can be obtained by rotating and inputting different mirror groups and the forward and reverse order of the input mirror group directions. 2 groups of mirror groups can obtain 4 magnifications ( 6 times, 25.6 times, 10 times, 64 times), plus 1 magnification ( 16 times ) can be obtained with the straight-through optical path without lens, and a total of 5 magnifications can be obtained.

Dry eye examination instruments based on slit lamps require different examination magnifications for various examinations of dry eye, such as: tear river height measurement, tear film break-up time measurement, lipid layer analysis using 16 magnifications ; Meibomian gland analysis 6 magnifications ; glands Body opening measurement, eye redness analysis 10 times. At this stage, the magnification switch of the non-automatic slit lamp type dry eye inspection instrument adopts the method of manually rotating the variable magnification objective lens. When the magnification is required, manually rotate the zoom handwheel to turn to that magnification. Its advantages are simple structure, direct and effective.

The existing proposed automation scheme of the slit lamp type dry eye inspection instrument only covers the automatic X, Y and Z direction movement of the platform; the automatic input and output of the dry eye illumination projection device, but does not include the optical magnification of the slit lamp body microscope Automatic switching, its problems are:

In the automatic dry eye inspection, if the magnification is not adjusted, only the low-magnification optical system can be used to inspect all items. Now, the observation of high-magnification items at low magnification will reduce the resolution of the image and affect the judgment of the results. and the realization of the result.

This patent is to solve the automatic zooming problem of the optical system zooming device of the Galileo slit lamp type dry eye inspection instrument, and is compatible with the manual zooming function of the original optical system zooming device. question.

### SUMMARY OF THE INVENTION

Aiming at the deficiencies of the prior art, the present invention proposes an automatic and manual magnification mechanism based on a slit lamp type dry eye inspection instrument . The specific technical scheme is as follows:
An automatic and manual zoom mechanism based on a slit lamp dry eye inspection instrument, characterized in that it comprises an optical body zoom drum seat, an optical body zoom drum, a zoom handwheel and an electric drive mechanism;
The optical body variable magnification drum is installed in the optical body variable magnification drum seat, and two ends of the optical body variable magnification drum are respectively connected with variable magnification handwheels through connecting shafts;
An electric drive mechanism is arranged on the optical body variable magnification drum base, and the electric drive mechanism is used to drive the optical body variable magnification drum to rotate to realize electric magnification.

Further, the electric drive mechanism may be a gear mechanism or a sheave mechanism or a pulley mechanism. Further, the electric drive mechanism may be a gear mechanism, a sheave mechanism, a pulley mechanism, or the like.

Further, the electric drive mechanism is that the motor directly drives the optical body variable magnification drum to rotate.

Further, the electric drive mechanism includes a motor, a driving gear and a driven gear, the motor is installed on the optical body variable magnification drum seat, the driving gear is installed on the output end of the motor, and the driven gear is installed on the output end of the motor. The movable gear is fixedly sleeved on the connecting shaft.

Further: the teeth on the driving gear are partial teeth, and the driving gear drives the driven gear to move intermittently through the partial teeth;

When the driving gear does not mesh with the driven gear, the driven gear and the connected optical body variable-magnification drum can rotate freely together.
The beneficial effects of the invention are as follows: first, the structure is simple, and it is convenient to realize the automatic magnification function of the slit lamp of the Galileo optical system and the slit lamp type dry eye inspection instrument;
Second, while realizing the automatic zoom function of the slit lamp of the Galileo optical system, the manual zoom function is also retained;

Third, it is convenient to realize the intelligent operation and remote operation of the entire slit lamp and the slit lamp type dry eye inspection instrument.

### DESCRIPTION OF DRAWINGS

FIG. 1 is the main sectional view of the patent structure;

FIG. 2 is a sectional view of the patent structure AA ;

FIG. 3 is a sectional view of the patented structure BB ;

FIG. 4 is an exploded view of the patent structure;

FIG. 5 is a schematic diagram of the patent structure;

FIG. 6 is the logic block diagram of the patent;

FIG. 7 is a schematic diagram of the optical body of the slit lamp Galileo system;

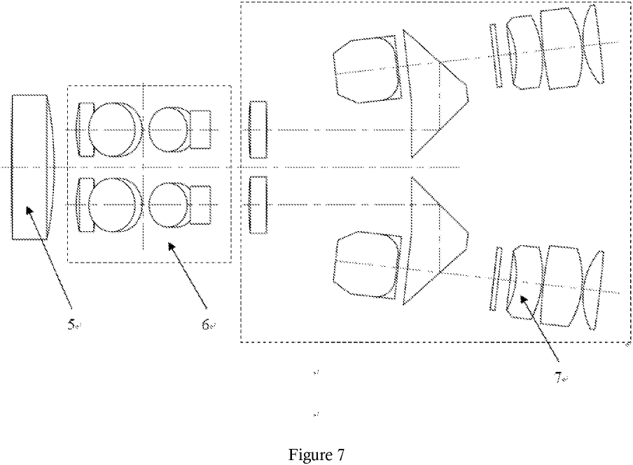

The description of the drawings is marked as: slit lamp chin rest mechanism 1, slit lamp illumination mechanism 1, microscope optical body 3, slit lamp platform moving mechanism 4, slit lamp Galileo system optical body large objective lens 5, slit lamp Galileo system optical body variable Multiplier objective lens 6, slit lamp Galileo system optical body eyepiece 7, optical body left zooming handwheel 301, left blocking plate 302, left adjusting ring 303, optical body zoom drum seat 304, large object seat 305, optical body zoom Rotary drum 306, driven gear 307, right blocking plate 308, optical body right zooming hand wheel 309, driving gear end cover 310, driving gear 311, motor 312, right shrapnel retaining ring 313, point bearing 314, mandrel 315, Left shrapnel retaining ring 316, shrapnel 317.

### DETAILED WAYS

The preferred embodiments of the present invention will be described in detail below with reference to the accompanying drawings, so that the advantages and features of the present invention can be more easily understood by those skilled in the art, and the protection scope of the present invention can be more clearly defined.

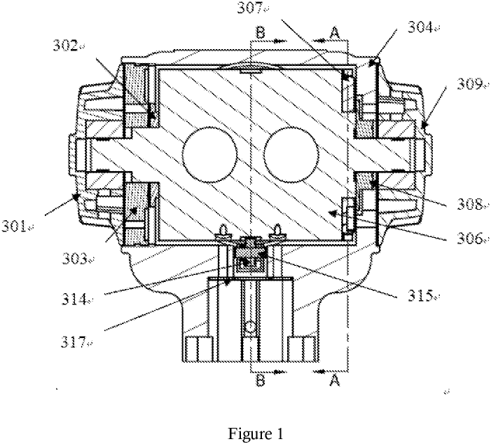

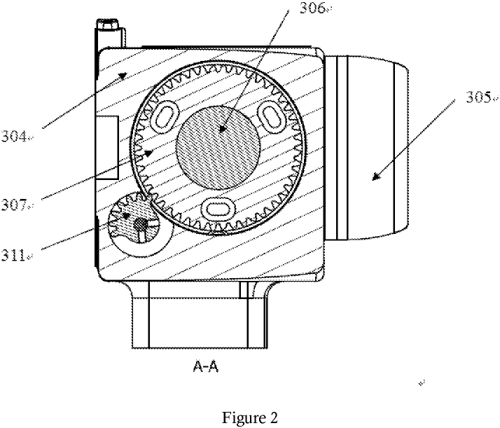

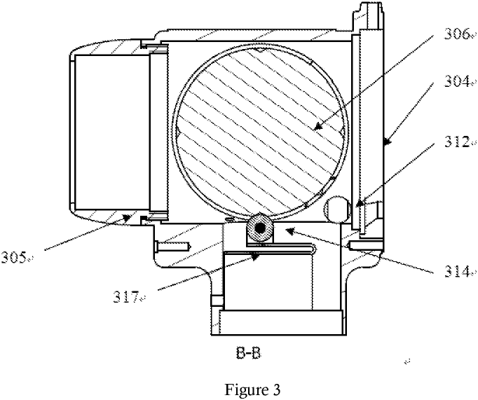

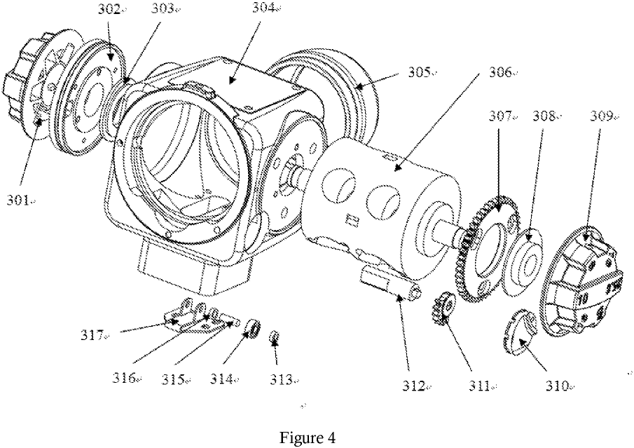

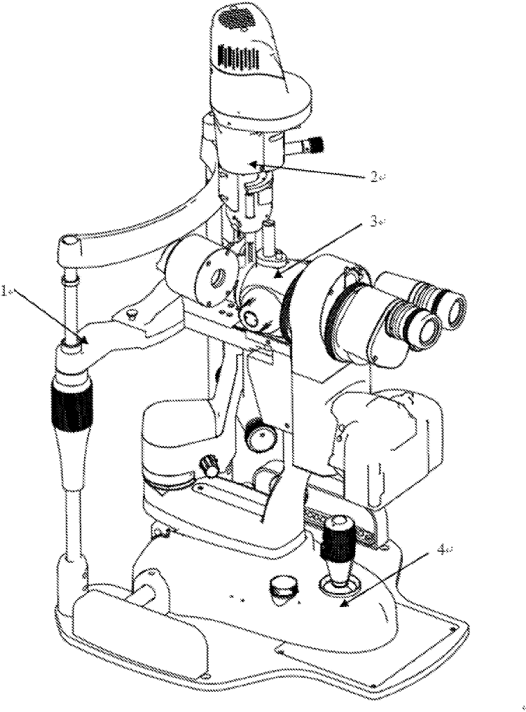

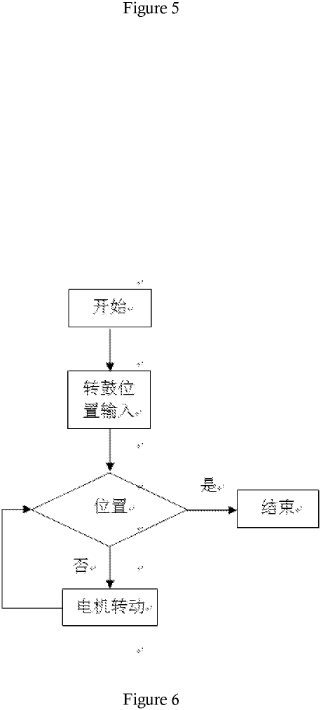

FIG. 1 to FIG. 6 : an automatic and manual zoom mechanism based on a slit lamp type dry eye inspection instrument, including an optical body zoom drum base 304, an optical body zoom drum 306, and an optical body left zoom The handwheel 301, the right zooming handwheel 309 of the optical body, the right blocking piece 308 and the left blocking piece 302.

306 is provided in the optical body variable magnification drum base 304, the left end of the optical body variable magnification drum 306 is provided with a left connecting shaft, and the right end of the optical body variable magnification drum 306 is provided with a right The connecting shaft, the left end of the left connecting shaft and the right end of the right connecting shaft respectively protrude from the corresponding side of the optical body variable magnification drum base 304, and the free end of the left connecting shaft is connected with an optical body left zooming handwheel 301, the free end of the left connecting shaft is connected with an optical body left zooming hand wheel 301, and the free end of the right connecting shaft is connected with an optical body right zooming handwheel 309, and a follower is respectively sleeved on the right connecting shaft The gear 307 and the right blocking piece 308, the right blocking piece 308 is located between the driven gear 307 and the right zooming handwheel 309 of the optical body;

on the optical body zooming drum base 304, a motor 312 is installed in the motor installation hole, and a driving gear 311 is connected to the output end of the motor 312, and the driving gear 311 has some teeth, In this embodiment, the driving gear 311 adopts seven teeth, and the driving gear 311 drives the driven gear to move intermittently.

driving gear 311 does not mesh with the driven gear 307, the driven gear 307 and the connected variable magnification drum 306 can rotate freely together.

At the bottom of the optical body variable magnification drum base 304 is a locking groove, and a locking mechanism is arranged in the locking groove. The locking mechanism includes a right elastic plate retaining ring 313, a point bearing 314, a mandrel 315, a left Shrapnel retaining ring 316 and shrapnel 317, the upper surface of the shrapnel 317 is provided with a mandrel 315 through the support seat, a point bearing 314 is sleeved in the middle of the mandrel 315, and the left end and the right end of the mandrel 315 are respectively provided with left shrapnel The retaining ring 316 and the right spring retaining ring 313. The point bearing 314 is used for clamping the optical body variable magnification drum 306. Grooves have been opened on the circumference of the optical body zoom drum 306. When the slot of the optical body zoom drum 306 rotates to the top of the point bearing 314, the elastic piece 317 drives the point bearing 314 to snap into the corresponding in the card slot.

The working principle of the present invention: part of the meshing teeth are removed from the driving gear (only some of the teeth are retained), when the driving gear rotates, only the remaining number of teeth participates in driving the driven gear to rotate, so when the driving gear rotates continuously, the driven gear rotates intermittently. sports.

When it is necessary to manually adjust the zoom drum of the optical body, the driving gear rotates to the non-mesh position, and the driven gear can rotate freely. The left zoom handwheel or the right zoom handwheel of the optical body drives the optical body zoom drum to rotate to the required position through the corresponding connecting shaft, and is locked into the slot on the zoom drum by the positioning bearing installed on the shrapnel achieve precise positioning.

When the optical body zoom drum needs to be adjusted automatically, the motor drives the driving gear to rotate, the driving gear drives the driven gear to rotate, and the driven gear drives the optical body zoom drum to rotate through the right connecting shaft. When the position is changed, the displacement drum just rotates the positioning position to achieve the same precise positioning as manual operation.

It will be apparent to those skilled in the art that the present invention is not limited to the details of the above-described exemplary embodiments, but that the present invention may be embodied in other specific forms without departing from the spirit or essential characteristics of the invention. Therefore, the embodiments are to be regarded in all respects as illustrative and not restrictive, and the scope of the invention is to be defined by the appended claims rather than the foregoing description, which are therefore intended to fall within the scope of the claims. All changes within the meaning and scope of the equivalents of, are included in the present invention. Any reference signs in the claims shall not be construed as limiting the involved claim.

In addition, it should be understood that although this specification is described in terms of embodiments, not each embodiment only includes an independent technical solution, and this description in the specification is only for the sake of clarity, and those skilled in the art should take the specification as a whole, the technical solutions in each embodiment can also be appropriately combined to form other implementations that can be understood by those skilled in the art.

1. An automatic and manual zooming mechanism based on ophthalmic slit lamp with dry eye inspection instrument, characterized in that: it comprises an optical body zooming drum base, an optical body zooming drum, a zooming handwheel and an electric drive mechanism;
the optical body variable magnification drum is installed in the optical body variable magnification drum seat, and two ends of the optical body variable magnification drum are respectively connected with variable magnification handwheels through connecting shafts;
an electric drive mechanism is arranged on the optical body variable magnification drum base, and the electric drive mechanism is used to drive the optical body variable magnification drum to rotate to realize electric magnification.

2. An automatic and manual zooming mechanism based on ophthalmic slit lamp with dry eye inspection according to claim 1, wherein the electric drive mechanism can be a gear mechanism, a sheave mechanism, or a pulley mechanism.

3. An automatic and manual zooming mechanism based on ophthalmic slit lamp with dry eye inspection according to claim 1, wherein the electric drive mechanism is a motor that directly drives the optical body to rotate the magnification changing drum.

4. An automatic and manual zooming mechanism based on ophthalmic slit lamp with dry eye inspection according to claim 2, wherein: the electric drive mechanism includes a motor, a driving gear and a driven gear, the motor is mounted on the optical body variable magnification drum seat, the driving gear is mounted on the output end of the motor, and the driven gear is fixed. sleeved on the connecting shaft.

5. An automatic and manual zooming mechanism based on ophthalmic slit lamp with dry eye inspection according to claim 4, wherein the teeth on the driving gear are partial teeth, and the driving gear drives the driven gear through the partial teeth. Intermittent movement of gears;
when the driving gear does not mesh with the driven gear, the driven gear and the connected optical body variable-magnification drum can rotate freely together.
